(19) United States
(12) Patent Application Publication
(10) Pub. No.: US 2008/0003660 A1
(43) Pub. Date: Jan. 3, 2008

(54) Use of fast-release pristinamycin-type and polyether ionophore type antimicrobial agents in the production of ethanol

(76) Inventors: Dennis P. Bayrock, Saskatoon (CA); Michael P. Pompeo, Sumter, SC (US)

Correspondence Address: HAYDEN STONE, PLLC, 101 NORTH COLUMBUS ST., SUITE 409, ALEXANDRIA, VA, 22301, US

(21) Appl. No.: 11/806,592

(22) Filed: Jun. 1, 2007

Related U.S. Application Data
(60) Provisional application No. 60/812,965, filed on Jun. 13, 2006.

(51) Int. Cl. C12N 1/20

(52) U.S. Cl. 435/252.9

(57) ABSTRACT

A method of controlling microorganisms such as lactobacilli metabolism in mash in an ethanol production facility includes adding to the mash an effective amount to control such microorganisms of one or more of a substantially water insoluble pristinamycin-type antimicrobial agent, a substantially water insoluble polyether ionophore antimicrobial agent, or both, wherein the term “substantially water insoluble” means the antimicrobial agent has a solubility in pure water at 20° C. of 0.1 grams per liter or less, and wherein at least a portion of the substantially water insoluble antimicrobial agent(s) is added to the mash in the form of particles comprising said substantially water insoluble antimicrobial agent(s) and having a weight mean average diameter of less than 5 microns. Particles having a weight mean diameter between 0.1 and 1 microns are preferred.

### CROSS-REFERENCE TO RELATED APPLICATIONS

This application claims priority to U.S. Provisional application No. 60/812,965 filed Jun. 13, 2006, the entire document of which is incorporated by reference herein for all purposes.

### STATEMENT REGARDING FEDERALLY SPONSORED RESEARCH OR DEVELOPMENT

N/A.

### INCORPORATION-BY-REFERENCE OF MATERIAL SUBMITTED ON A COMPACT DISC

N/A.

### SEQUENCE LISTING

N/A.

### FIELD OF THE INVENTION

The present invention relates to the use of delivery systems to deliver measured quantities of antimicrobial agents, and particularly pristinamycin-type antimicrobial agents or polyether ionophores, to industrial processes, particularly to processes involving the alcohol production via fermentation, in a form where such antimicrobial agents are available to the fluid immediately or in a short period of time. The antimicrobial agent are added in the form of very small particles that are advantageously less than 5 microns in diameter and preferably less than 1 micron in diameter. Sub-micron particles quickly provides available biocidal activity even in poorly stirred reaction vessels where traditional powdered biocidal agents are substantially ineffective.

### BACKGROUND OF THE INVENTION

Ethanol production through anaerobic fermentation of a carbon source by the yeast Saccharomyces cerevisiae is one of the best known biotechnological processes and accounts for a world production of more than 35 billion liters per year. Two thirds of the production is located in Brazil and in the United States with the primary objective of using ethanol as a renewable source of fuel. Hence, there are strong economic incentives to further improve the ethanol production process. The price of the sugar source or carbohydrate source is a very important process parameter in determining the overall economy of ethanol production. Using unaltered yeasts, the greatest yield obtainable is only about 51.1%, with the remainder being lost to yeast maintenance and growth, glycerol production, and other end products. Hence, it is of great interest to optimize the ethanol yield in order to ensure an efficient utilization of the carbon source. The typical ethanol yield is lower than the above-described maximum theoretical yield in large part due to competing microorganisms.

A typical ethanol production plant comprises a premixing vessel where water and the carbohydrate fuel source (hereafter referred to as mash) are held at 40° C. to 60° C. and where (if corn is the source of carbohydrate) a small amount of enzyme such as a-amylase is added. The mash is then heated to between 90° C. to 150° C. for a period of time, and then cooled and held between 80° C. to 90° C. as the mash liquifies. The mash is then cooled to 60° C. and additional enzymes may be added in a saccharification step. After a period of time at 60° C., the mash is cooled to ambient to ˜35° C., and the liquid is then sent to fermenters where yeast is added to convert sugars to ethanol. In a continuous process utilization of a number of serially linked fermenters is typical, as this is required for efficient conversion of the sugars and also because ethanol-production-favorable conditions (which depend on the amount of alcohol and other byproducts present in the mash) can be optimized. Finally, the alcohol/water fraction is sent to a distilling column where alcohol is extracted, and the residual material find large markets in the animal feed business. Large volumes are processes, and as one might imagine with all the temperature changes involved in the process that heat exchangers are critical to both net production of energy and to the economics of the process.

One particularly difficult problem is the control of competing microorganisms, in particular Lactobacillus spp., which compete with the yeast for nutrients and produce lactic acid. Other microorganisms such as Acetobacter/Gluconobacter and wild yeasts must also be controlled. Since control of lactobacilli is more critical to the process viability and since control of one class of microorganisms by the methods described here results in control of at least some of the other microorganisms, this discussion will focus on lactobacilli control. One of skill in the art will know that a number of other competing microorganisms will also be controlled by the treatment processes described here, depending on the antibiotics and antimicrobials used in the process. Lactobacilli contamination in the range of 10⁶ to 10⁷ per ml can reduce ethanol yield by 1-3%. Lactobacilli are present in all incoming carbohydrate sources, and are present in all areas of the ethanol production plant. In industrial processes such as the manufacture of ethanol for fuel, even with active control programs to control the proliferation of lactobacilli, carbohydrate losses to lactobacilli can range up to several percent of the total carbohydrate input, which can make the difference between profitability and non-profitability. Further, if the lactic acid content of the mash approaches 0.8% and/or acetic acid concentration exceeds 0.05%, the ethanol producing yeast are stressed and yeast metabolism is reduced. In the manufacture of certain alcoholic beverages, the proliferation of lactobacilli and its byproducts can unfavorably alter the taste and value of the product.

One very effective control program involves the introduction of pristinamycin-type antimicrobial agents, and particularly virginiamycin, to the process. These pristinamycin-type antimicrobial agents, and particularly virginiamycin, are preferred because: 1) they are very effective against a number of microorganisms including lactobacilli at low concentrations, e.g., 0.3 to 5 ppm, 2) microorganisms do not tend to develop resistance to this type of antimicrobial agent, 3) the antimicrobial agent does not significantly hinder the yeast, and 4) the antimicrobial agent is effectively destroyed by the drying of the end “waste” product so that it is not introduced indiscriminately into the environment. Usually, the “waste” byproduct, known as “Dried Distillers Grains with solubles (DDGS), is sold as animal feed, going 45% to dairy, 35% to beef, 15% to swine, and 5% to poultry industries. This is an important factor in the profitability of an ethanol production process, and the total amount of this byproduct produced per year is on the order of 3.5 million metric tons per year. The presence of residual antimicrobial agents in this material can adversely affect the value of this byproduct, as small residual amounts of antimicrobial agents in feed will promote the development of agent-resistant microorganisms. We have tested DDG samples from 8 major ethanol producers using virginiamycin to control microorganisms and found no detectable amount of virginiamycin in the DDG (<1 ppm via the validated Eurofins analysis and <1 ppb via an unvalidated experimental analytical procedure). Incidentally, animal feed is often supplemented with virginiamycin, which has been shown to significantly increase production when used in a number of animal feeds. Generally, however, the virginiamycin in mash is destroyed by drying so virginiamycin must be re-added to the feed if so desired. Other effective control programs employ polyether ionophore antimicrobial agents, which provide many of the benefits obtained with pristinamycin-type antimicrobial agents. Other control agents used in the industry include tetracycline-based antibiotics, streptomycin, penicillin-based antibiotics (e.g., G, V, or N), and bacitracin. These are not favored because microorganisms can quickly develop tolerances and presence of microorganisms that are resistant to these antibiotics can create problems with the public perception and with some uses of the waste or residual material after fermentation as animal feed. In tests with virginiamycin, a mixture of ˜70-75% penicillin/10-15% virginiamycin/10-15% streptomycin, and “KPenG” a commercial product, we found L. plantarum developed resistance to KPenG in about 2 weeks, and developed resistance to the mixture in about a week, but showed no development of resistance to virginiamycin over the entire 10 week duration of the test. Further, penicillin and streptomycin are partially inactivated at the pH in the fermenters. Also, there are issues with worker safety and allergies.

It has been demonstrated that for antibiotics such as penicillin that pulsed addition of antibiotics is significantly superior compared to continuous addition of the same amount of antibiotic. See, e.g., Control of Lactobacillus contaminants in continuous fuel ethanol fermentations by constant or pulsed addition of penicillin G, Appl Microbiol Biotechnol (2003) 62:498-502 by Bayrock, Thomas, and Ingledew. This is believed to extend to other types of antimicrobial agents. We have tested pulsed dosing versus continuous dosing on L. paracasei and found pulse dosing lowered the microorganism count to about 30% of the value obtained with continuous dosing, where the same amount of antimicrobial agent is added in both cases. It is generally known that higher concentrations of antimicrobial agents result in higher numbers of targeted microorganisms being destroyed than are destroyed at lower concentrations. Pulsed mode addition of antimicrobial agents is believed to be more effective than continuous treatment because the higher concentration (even if present for only a short time) reduces the number of targeted microorganisms sufficiently that the rebound of surviving targeted microorganisms during periods between treatments results in fewer total viable microorganisms (averaged over time) than are obtained by continuous treatment with the same quantity of antimicrobial agent.

The processes and materials of this invention are particularly useful to introduce antimicrobial agents having very low solubility in water, e.g., a solubility of less than about 10⁻² and often less than about 10⁻³ grams per liter in water. The solubility of monensin, virginiamycin, and similar pristinamycin-type antimicrobial agents and polyether ionophore-type antimicrobial agents in water is very low. Pristinamycin-type antimicrobial agents, especially virginiamycin, have extremely low solubility in water (e.g., 0.0001 grams/l), and additionally the kinetics of dissolution are very poor. Similarly, polyether ionophores have extremely low solubility in water.

The over-riding factor in controlling pests such as lactobacilli, however, is the rate of dissolution of small granular pristinamycin-type antimicrobial agents and polyether ionophore-type antimicrobial agents in water or mash. A 0.1 gram sample of a 5.2 to 10 micron average particle size virginiamycin was placed in a beaker with 4 liters of water, and the composition was continuously and vigorously stirred (providing very good mixing and turbulence). The presence of undissolved crystals was very evident. It took on the order of an hour before only a few crystals of the material remained visible. Such slow dissolution will reduce effectiveness of pulse treatments as it takes a long time for the added agents to become solubilized and effective, and will reduce the highest concentration of added agents resulting from a pulsed addition as some of the agent may be removed from the fermentator or other tank before the maximum amount of added agent is solubilized, and because some added agent may not dissolve at all.

The typical treatment of ethanol plants with pristinamycin-type antimicrobial agents or polyether ionophores is provided by intermittently adding powders either as loose material or encased in dissolvable bags or packets containing a predetermined amount of the antimicrobial agent to one or more of the large mixed tanks. Two commercial prior art formulations used in ethanol treatment plants of virginiamycin comprised powder of average diameter of 5.2 microns and about 1000 microns. In these large mixed tanks, there is often sufficient residence time and mixing for some portion of the virginiamycin to dissolve. However, mash vats and other large tanks in ethanol production plants typically are not rigorously and completely stirred, as the energy needed for such mixing can outweigh small gains in the yeast efficiency. In a poorly mixed environment, we have determine dissolution rates can take many hours, and some fraction of a granular pristinamycin-type antimicrobial agent and/or polyether ionophore-type antimicrobial agent product may never be solubilized and thereby activated.

In a smaller ethanol production plant (where the product is a distilled beverage), even introduction of virginiamycin in ˜5+ micron powdered form into vigorously stirred mixing tanks does not result in complete dissolution of the antimicrobial agent, and solid antimicrobial agent material that does not dissolve is wasted.

### SUMMARY OF THE INVENTION

The invention can be broadly described as a method of controlling lactobacilli metabolism in mash in an ethanol production facility, comprising adding to the mash an effective amount of one or more of a substantially water insoluble pristinamycin-type antimicrobial agent, a substantially water insoluble polyether ionophore antimicrobial agent, or both, wherein the term “substantially water insoluble” means the antimicrobial agent has a solubility in pure water at 20° C. (ambient) of about 0.1 grams per liter or less, and wherein the substantially water insoluble antimicrobial agent(s) is added to the mash in the form of particles comprising or consisting essentially of said substantially water insoluble antimicrobial agent(s) and having a weight mean average diameter of less than 5 microns, preferably less than 2 microns, more preferably less than 1 micron, for example between 0.1 and 1 microns. Advantageously at least 50% by weight, preferably at least 70% by weight, more preferably at least 90% by weight, of the added antimicrobial agent is in particles having a diameter of less than 5 microns, preferably less than 2 microns, more preferably less than 1 micron, for example between 0.1 and 1 microns.

In a preferred embodiment the substantially water insoluble antimicrobial agent comprises or consists essentially of at least one of virginiamycin and semduramycin and at least a portion of the antimicrobial agent(s) is added to the mash in the form of particles comprising said substantially water insoluble antimicrobial agent(s) and having a weight mean average diameter of less than 2 microns. In another embodiment the substantially water insoluble antimicrobial agent comprises or consists essentially of monensin and at least a portion of the monensin is added to the mash in the form of particles comprising monensin and having a weight mean average diameter of less than 2 microns. In another embodiment the substantially water insoluble antimicrobial agent comprises or consists essentially of a substantially water insoluble pristinamycin-type antimicrobial agent. In another embodiment the substantially water insoluble antimicrobial agent comprises or consists essentially of a substantially water insoluble polyether ionophore antimicrobial agent.

The powders of this invention can advantageously be wetted with an organic liquid comprising at least one of alkyl acetate where the alkyl moiety has between 1 and 4 carbon atoms, alkyl lactate where the alkyl moiety has between 1 and 4 carbon atoms, N,N-dialkylcapramide where the alkyl moiety has between 1 and 4 carbon atoms, dialkylsulfoxide where the alkyl moiety has between 1 and 4 carbon atoms, N-alkylpyrrolidone where the alkyl moiety has between 1 and 4 carbon atoms, pyrrolidone, dialkyl formamide where the alkyl moiety has between 1 and 4 carbon atoms, acetone, isopropanol, a butanol, a pentanol, or combinations thereof. Such wetting should be done immediately prior to adding the powders of this invention to the mash. Preferred wetting solvents include dipolar aprotic organic solvents, alkyl acetate, alkyl lactate, particularly ethyl acetate or ethyl lactate, or combination thereof. Preferred aprotic solvents include alkyl pyrrolidone, an amide, or a dialkylsulfoxide. Advantageously if the powders of this invention are wetted with a liquid comprising an organic solvent prior to adding the powder to mash, the wetting liquid has a closed cup flash point of greater than 200° F.

In another embodiment at least a portion of said antimicrobial agent is added to the mash as a composition comprising particles comprising said substantially water insoluble antimicrobial agent(s) and having a weight mean average diameter of between 0.1 and 1 microns. Of course, this invention includes powders where almost half of the weight of the added antimicrobial agent powder has a diameter less than 5 microns, and preferably less than 2 microns, which would be obtained by adding a product of this invention along with a prior art powdered formulation, thereby raising the “measured” weight mean average particle diameter to greater than 5 microns, as the particles of this invention will provide the described benefits and that most of said larger particles will eventually dissolve and give some additional benefit. Therefore, this invention also encompasses such treatments, where at least a third of the weight of the particles added in a treatment have a particle diameter less than 5 microns, preferably less than 2 microns, for example between 0.1 and 1 microns.

Additionally, the particle size is to be measured after particles are added to water, as surfactants, sugars, and other such materials rapidly dissolve. In another embodiment at least a portion of said antimicrobial agent is added to the mash as a composition comprising particles comprising said substantially water insoluble antimicrobial agent(s) and having a weight mean average diameter of less than 5 microns, said particles being enveloped in a solid inert medium having a composite particle size greater than 5 micron or in a grease-like inert medium, said inert medium being selected to provide rapid dissolution in the mash and subsequent dispersion of said particles in the mash such that the particles are dispersed in the mash within two minutes of adding the composition to the mash. Such inerts include alkali containing carbonates such as sodium bicarbonate, alkali containing phosphates, detergents or surfactants, and the like. In another embodiment at least a portion of said antimicrobial agent is added to the mash as a composition comprising particles comprising said substantially water insoluble antimicrobial agent(s) and having a weight mean average diameter of between 0.1 and 2 microns, said particles being enveloped in a solid inert medium having a particle size greater that 5 micron or in a grease-like inert medium, said inert medium being selected to provide rapid dissolution in the mash and subsequent dispersion of said particles in the mash such that the particles are dispersed in the mash within two minutes of adding the composition to the mash.

In another embodiment at least a portion of said antimicrobial agent is added to the mash as a composition comprising particles comprising said substantially water insoluble antimicrobial agent(s), said composition being in the form of a slurry. In another embodiment at least a portion of said antimicrobial agent is added to the mash as a composition comprising particles comprising said substantially water insoluble antimicrobial agent(s) and having a weight mean average diameter of between 0.1 and 2 microns, said composition being in the form of a slurry. Many of the preferred antimicrobial agents have a slight instability when dissolved in water, which can be significant over long storage periods. Virginiamycin, for example, appears to be subject to slow hydrolysis when in water. Coating particles with protectorants will reduce stability problems. Placing the slurry in a non-organic substantially water-free material, be it fatty acids, surfactants, dispersants, solvents in which the antimicrobial agents have minimal solubility (called an “oil flowable slurry”), or any combination of the above can reduce loss of antimicrobial agent. For example, in another embodiment at least a portion of said antimicrobial agent is added to the mash as a composition comprising particles comprising said substantially water insoluble antimicrobial agent(s), said composition being in the form of a slurry further comprising water and trehalose. In yet another embodiment at least a portion of said antimicrobial agent is added to the mash as a composition comprising particles comprising said substantially water insoluble antimicrobial agent(s), said composition being in the form of a slurry further comprising a solvent in which the antimicrobial agents have less than 1 gram/liter solubility, preferably less than 0.1 grams/liter solubility, more preferably less than 0.01 grams/liter solubility. Protectorants such as trehalose can be added to the particles in an oil flowable slurry, though the loss due to hydrolysis will be sharply reduced in an oil flowable slurry as compared to losses of antimicrobial agents in an aqueous slurry. Advantageously the liquid phase of an oil-flowable slurry comprises solvents having some modest solubility in water, e.g., at least 0.1 g/l, to help dissipate droplets of the injected slurry into the mash. An oil-flowable slurry can be readily prepared by milling the antimicrobial agents as described herein, but where the solvent replaces the water in the milling process. Or, the antimicrobial agent can be milled in water, and then the water be removed by drying or washing with solvent. As an alternative to a slurry, which we define as a liquid having particles suspended therein, the particles can be encased in a solid or semisolid material comprising mono, di, or triglycerides of fatty acids, fatty acids, surfactants, dispersants omega-3 fatty acids, DHA, docosapentaenoic acid, tetracosapentaenoic acid, tetracosahexaenoic acid, monounsaturated fatty acids, polyunsaturated fatty acids, saturated fatty acids, trans fatty acids, derivatives thereof, and mixtures thereof, where the encasing material is dispersible and is preferably soluble in the mash in the injected amounts.

In many instances the antimicrobial agent is added to mash, to water, or to another process stream which is at an elevated temperature, e.g., greater than 35° C. for example. In such a case advantageously the encasing material may be a water free or substantially water free (less than 10% by weight water) solid at ambient temperature but softens or melts at a slightly elevated temperature such as 35° C., for example. In any and each of the above-described embodiments, advantageously the antimicrobial agent comprises virginiamycin, and at least a portion, and preferably at least one half by weight, of said virginiamycin is added to the mash as a composition comprising particles comprising said virginiamycin and having a weight mean average diameter of between 0.1 and 0.7 microns.

In another embodiment the ethanol production facility comprises at least one mixed tank and at least one heat exchanger, the method comprising: a) adding to the mash in said tank a portion of the substantially water insoluble antimicrobial agent(s) in the form of particles comprising said substantially water insoluble antimicrobial agent(s) and having a weight mean average diameter of less than 5 microns; and b) adding to the mash passing through said heat exchanger a portion of the substantially water insoluble antimicrobial agent(s) in the form of particles comprising said substantially water insoluble antimicrobial agent(s) and having a weight mean average diameter of less than 2 microns.

In another embodiment the invention is a method of controlling lactobacilli metabolism in mash in an ethanol production facility, comprising adding to the mash an effective amount of one or more of a substantially water insoluble pristinamycin-type antimicrobial agent, a substantially water insoluble polyether ionophore antimicrobial agent, or both, wherein the term “substantially water insoluble” means the antimicrobial agent has a solubility in pure water at 20° C. of 0.1 grams per liter or less, and wherein at least a portion of the substantially water insoluble antimicrobial agent(s) is added to the mash in the form of particles comprising said substantially water insoluble antimicrobial agent(s), wherein at least one third of the total weight of said particles added in a treatment have a weight mean average diameter of less than 5 microns, preferably less than 2 microns, for example between 0.1 and 2 microns.

The formulations discussed above are useful for a variety of applications in addition to controlling undesired microorganisms in ethanol production facilities. Antimicrobial agents such as virginiamycin are used in a large number of applications, including the above-mentioned use as a supplement given to animals to encourage growth. The powders and slurries of various embodiments of this invention are equally applicable to use in those fields of use, providing a number of benefits including reduced dust, easy incorporation of antimicrobial agents into feed, and greater stability and dispersability in water systems.

### BRIEF DESCRIPTION OF THE DRAWINGS

The drawings show data from a number of experiments as described below:

FIG. 1 shows the Lactobacillus count versus time in mash from the well-mixed fermentators treated with Lactrol™ (Brazil) brand virginiamycin, Lactrol™ (Belgium) brand virginiamycin, virginiamycin solubilized in dimethylsulfoxide (DMSO) according to this invention, and also the Lactobacillus count in a well-mixed control fermentator.

FIGS. 2 and 3 show (for duplicate experiments) the Lactobacillus count versus time in mash from the poorly-mixed fermentators treated with Lactrol™ (Brazil) brand virginiamycin, Lactrol™ (Belgium) brand virginiamycin, virginiamycin solubilized in DMSO according to this invention, and in a poorly-mixed control fermentator.

### DETAILED DESCRIPTION OF PREFERRED EMBODIMENTS

Typically, disclosures herein center on virginiamycin, as that is the preferred antimicrobial agent. It should be appreciated, however, that these disclosures are also generally applicable to other pristinamycin-type antimicrobial agents and polyether ionophore-type antimicrobial agents.

Another aspect of this invention is providing antimicrobial agents, particularly pristinamycin-type antimicrobial agents, polyether ionophore-type antimicrobial agents, or both, to mash or to an ingredient forming the mash in an ethanol production plant, where the antimicrobial agents are added in the form of a powder having a weight mean average diameter of less than 10 microns, preferably less than 5 microns, more preferably less than 2 microns, for example having a weight mean average diameter of between 0.1 microns and 1 microns, in a continuous mode, in a pulsed mode, or in some alternative hybrid mode. Use of such a small diameter provides a number of advantages over the prior art formulations, which used for example particles having a diameter of over 10 microns. We have found that prior art formulations do not provide the anticipated concentration profile when admixed into tanks, as it takes a long period of time (more than 10 minutes) for such particles to fully dissolve in aqueous mash, and the hydrodynamic conditions and residence time of the particles in the mixing tank are such that some of the antimicrobial material will not dissolve but will be effectively wasted. Therefore, a pulse treatment using prior art powdered antimicrobial agents having particle diameters above 5 microns in a mixed tank in fact does not provide an active concentration of material as is often depicted in literature, that is, reaching a peak which subsequently declines as the pulse or dose is diluted by untreated incoming mash. Rather, the concentration of effective antimicrobial agents in a dosed mixed tank using prior art treatments tends to climb slowly and peaks at a point where a significant amount of the material has already left the mixed tank, and the peak concentration and the area under a concentration-time curve will both be much lower than anticipated. Adding the material in the form of particles of very highly reduced size will allow the material to be more dispersed in the liquid in a short period of time, and will allow the particles themselves to have a greatly increased rate of dissolution. Dissolution rates, in terms of mg active ingredient dissolved per liter of mash, can be over 100 times faster for a given weight of 0.5 micron particles as compared to the dissolution rate of the same weight of particles present as 5 micron particles. The faster dissolution rates allow several treatment regiments with pristinamycin-type antimicrobial agents, polyether ionophore-type antimicrobial agents, or both that were not possible with prior art formulations. First, the effective dose (that is, the dose of antimicrobial agent that is effectively used to control targeted microorganisms) more nearly matches the theoretical dose. Second, higher effective concentrations (and therefore increased efficacy) of biocide are achieved from a pulse dose of fast-dissolving particles than is obtainable with the same mass of slow dissolving particles. Third, tailoring a pulse in terms of effective concentration versus time and the duration of a pulse can be achieved. Fourth, the fast dissolving products of this invention can be utilized to pulse treat unit operations such as heat exchangers and small mixed tanks (especially for example saccharization tanks) where treatment with prior art formulations was not practical or possible because much of the added product would be flushed from the targeted unit operations prior to dissolution. Finally, fifth, the targeted bacteria have an effective diameter of about a micron. If the antimicrobial agent is of a size near that of a bacteria, say between ˜0.02 microns to ˜2 microns, a measure of control is obtainable from direct solid antimicrobial agent to microorganism contact and/or interaction, thereby increasing the efficacy of a mixture of soluble and particulate biocide of the current invention as compared to the efficacy of a mixture of soluble and particulate biocide of the prior art formulations.

A highly preferred particle size is a formulation of narrow particle size distribution distributed about a weight mean average of between 0.1 microns and 0.7 microns. It is highly advantageous that the particle size distribution be narrow. If a product has 1000 particles of diameter 0.5 microns and 1 particle of diameter 5 microns, then half of the weight of the product is present in the larger diameter particles. A preferred method of defining a narrow distribution is the d80 and d90, defined here as the diameter at which 80% and 90%, respectively, by weight the total antimicrobial agent present is in the form of particles having an effective diameter equal to or less than the d80 and d90, respectively. The weight mean average is the d50, that is, the diameter where half of the weight of the antimicrobial agent is present in the form of particles having an effective diameter of the d50 or less. In preferred formulations, the d80 and/or the d90 are within a factor of four, more preferably within a factor of three, and optimally within a factor of two of the d50.

That is not to say that there are no drawbacks of using very small particle size antimicrobial agents. The most significant drawback is the possibility of dust, both from normal operations and from normal shipping and handling of product. Submicron particles can act much like smoke or dust in the air. One method of controlling or eliminating accidental releases of submicron antimicrobial agents is to have these particles be contained in a slurry. A second method is to have the particles be encased in a dissolvable container that is impervious to the particles. This is not preferred as plant personnel may wish to break open a container to obtain a portion of a dose for one reason or another, and the remaining product will have a strong tendency to become airborne. A third mechanism of controlling dust from submicron particles of antimicrobial agents is to formulate these particles into a solid granular material, where the binding agent comprises for example a fast-dissolving sugar or salt matrix such as sodium bicarbonate. This granular material may further be placed in dissolvable containers for an added layer of prevention. Finally, the submicron particles may be contained in a semisolid material such as in a surfactant, e.g., in a 20 mole ethoxylated cocoamine surfactant material fatty acids, dispersants, mono-, di-, and/or tri-glycerides of fatty acids, and the like, or any combinations thereof. Each of these containment measured has the added benefit of allowing ready determination via weight or volume of the amount of antimicrobial agent added to a system.

Another aspect of this invention is therefore to supply a slurry comprising at least 0.1%, preferably at least 0.5%, for example at least 2% by weight of micron to sub-micron particles comprising or consisting essentially of one or more pristinamycin-type antimicrobial agents, polyether ionophore-type antimicrobial agents, or both, which are suspended in a liquid medium such as water, to mash or to an ingredient forming the mash in an ethanol production plant, where the antimicrobial agents are added to the mash in a continuous mode, in a pulsed mode, or in a hybrid mode. Advantageously the slurry will comprise at least 5%, more preferably at least 10%, for example between about 10% and 25% by weight of active antimicrobial agent in particulate form suspended or dispersible in a continuous liquid phase. The liquid phase may be aqueous, non-aqueous, water-free, or substantially water free (less than 10% by weight water based on the weight of the liquid phase), and may further comprise one or more additional soluble antibiotics.

We have found that wet milling virginiamycin in water with surfactants with a sub-millimeter zirconium-based ceramic milling medium can readily reduce virginiamycin to a weight mean average particle size of between 0.2 and 0.5 microns, but that at concentrations in excess of 10% by weight virginiamycin the slurry became “slushy” and viscous. While a slushy slurry is useful in treating mash in ethanol plants, insofar as it is readily measured and added to tanks, additives such as trehalose may advantageously be added to reduce this phenomenon. The production of monensin particles of diameter between 0.16 microns and 0.2 microns has been described in literature pertaining to anticancer treatments. The weight average particle size can vary between about 0.05 microns to about 10 microns, but is preferably below 5 microns, more preferably in the range of about 0.1 microns to about 2 microns, for example from about 0.1 microns to about 0.5 microns. These particles, preferably submicron particles, may additionally or alternatively comprise one or more other substantially insoluble antimicrobial agents. By substantially insoluble antimicrobial agents we mean for example (but not limited to) antimicrobial agents that have on a weight per volume basis a solubility within about a factor of fifty of the solubility of virginiamycin in that same medium.

For a formulation of submicron particles to be stable over a time frame of manufacturing, storing, shipping, and eventual use, it is important that the antimicrobial agents be stable and that the particle size and dissolution characteristics not be affected. This is easily achieved by using a dry formulation of “micron to sub-micron particles,” that is, a mass of particles having a particle size distribution such that the weight mean average particle size (diameter) is below 5 microns, preferably below 2 microns, for example between 0.05 microns to about 1 micron, preferably between 0.1 microns and 0.7 microns. The particles may be in the form of free individual particles of antimicrobial agent (encased in a container to reduce risk of dust and accidental release), where said particles may be treated with dispersants, or as agglomerated particles where the agglomerating medium is a fast-dissolving substance. Such dry particles are typically very stable. Generally, encasing the particles in a grease-like or oil-like enclosing medium, especially a water-free grease-like substance in which the antimicrobial agent has low solubility, will also stabilize the particles. Particle dissolution will be hindered by the viscosity of the medium, as well as by the limits of solubility of the antimicrobial agent in the medium. Most preferred antimicrobial agents used in this invention have a strongly polar character, and have solubility in strongly polar solvents. Therefore, solubility of the particles is minimized if the enclosing medium is has a non-polar character. However, this grease-like or oil-like medium can not overly hinder particle dissolution in the mash, or gains in dissolution rate obtained by the smaller particles may be off-set by the dissolution-hindering effect of the enclosing medium. Generally, the enclosing medium should have some level of solubility in the mash, that is, a solubility in mash at least equal to that of the antimicrobial agent. This tradeoff between nonpolar character to reduce dissolution and polar character to encourage coating dissolution in the mash is best met by fatty acids, ethoxylated surfactants, and the like. The problem of antimicrobial agent particle stability is particularly acute when the particles are shipped and stored as a slurry. Advantageously, unless the dose is formulated immediately before adding the dose to the mash, the liquid portion of the slurry should not dissolve more than a negligible amount of the antimicrobial agents. Slurries of submicron particles will tend to undergo dissolution from smaller particles and precipitation onto larger particles, which results in particle size growth over time. This growth rate is roughly proportional to the solubility of the antimicrobial agents in the liquid phase of the slurry. Again, there is a tradeoff between the polar character of the liquid (which encourages particle dissolution) and the dispersibility of the liquid medium in the mash. However, as with surfactants, when evaluating the solubility/dispersibility of the liquid medium, the total amount of this medium that would be added to mash by delivery of antimicrobial agents is on the order of 100 ppm or less, typically 20 ppm or less. Certain particle treatments, with for example surfactants, trehalose, and the like can be used to further inhibit antimicrobial agent solubility in the liquid portion of the slurry.

Generally, a preferred slurry from the standpoint of handlability and negligible effect on the mash and yeast is attained when the liquid phase of the slurry comprises or consists essentially of water. However, many of the preferred antimicrobial agents have a slight instability in water. Virginiamycin, for example, appears to be subject to slow hydrolysis when in water. Coating particles with protectorants such as oil, trehalose, dispersants, fatty acids, or combinations thereof to further isolate the particles of antimicrobial agents from the water will reduce stability problems. For example, in another embodiment at least a portion of said antimicrobial agent is added to the mash as a composition comprising particles comprising said substantially water insoluble antimicrobial agent(s), said composition being in the form of a slurry further comprising water and trehalose. However, these coatings must be disrupted or be dispersible so that the particles once injected into the mash will quickly dissolve.

As mentioned above, placing the slurry in a non-organic substantially water-free material, be it fatty acids, surfactants, dispersants, solvents in which the antimicrobial agents have minimal solubility (called an “oil flowable slurry”), or any combination of the above can reduce loss of antimicrobial agent to hydrolysis, as well as minimize particle growth during shipping and storage.

In yet another embodiment at least a portion of said antimicrobial agent is added to the mash as a composition comprising particles comprising said substantially water insoluble antimicrobial agent(s), said composition being in the form of a slurry further comprising a solvent in which the antimicrobial agents have less than 1 gram/liter solubility, preferably less than 0.1 grams/liter solubility, more preferably less than 0.01 grams/liter solubility. Protectorants such as trehalose, surfactants, dispersants, and the like can be added to the particles in an oil flowable slurry, though the loss due to hydrolysis will be sharply reduced in an oil flowable slurry as compared to losses of antimicrobial agents in an aqueous slurry. Advantageously the liquid phase of an oil-flowable slurry comprises solvents having some modest solubility in water, e.g., at least 0.1 g/l, to help dissipate droplets of the injected slurry into the mash. Suitable solvents for an oil-flowable slurry include ethers, alkanes, ketones, and the like not having a strongly polar character. An oil-flowable slurry can be readily prepared by milling the antimicrobial agents as described herein with water and advantageously surfactants, but where the solvent replaces the water in the milling process. Or, the antimicrobial agent can be milled in water as described herein, and then the water subsequently be removed by drying or washing with solvent. If an oil-flowable formulation is desired, a solvent having very low tendency to dissolve the antimicrobial agent, for example a petroleum ether, may be used. The slurry may be stored as a stable slurry, or as a water-mixable powder or as a settled slurry that may be admixed as needed, for example in a small high mixing and pumping unit capable of imparting sufficient shear to the materials so that the particles are effectively dispersed in the slurry. Such a slurry composition reduces the time necessary to get particles dispersed into the mash to a negligible value, and the reduced particle size (especially compared to previous treatments) facilitates dissolution of the particles.

Advantageously, the slurry comprises trehalose in an amount sufficient to reduce the dissolution rate if the antimicrobial agents in the liquid phase. Trehalose tends to coat lipid-like materials, and is useful both to stabilize slurries and as an additive to reduce agglomeration if the particles are freeze-dried or aerosol-dried to form a dried product. Trehalose has been used to stabilize submicron particles of monensin used to carry anticancer treatments, for example, during the freeze drying process. Further, trehalose is naturally occurring in ethanol fermentation processes and is utilized by yeast as a food source.

The slurry may alternatively or additionally comprise one or more thickeners, e.g., polyacrylate or guar gum, one or more dispersants, e.g., polyethylene glycol/poly(DL lactide glycolide diblock copolymers, carboxymethylcellulose, guar gum, and the like in an amount effective reduce the settling rate of the particles in the slurry.

It is advantageous to admix a slurry of particles comprising the antimicrobial agent under high shear or other specialized conditions to enhance dispersement and dissolution of particles. If the material comprising the antimicrobial agents is a solid or semisolid material comprising substantially water-free (less than 10% by weight water based on the weight of the material) fatty acids, surfactants, dispersants, oils, and the like, admixing with a small sidestream of mash or water under high shear will also aid dispersing and dissolving of the particles. This mixing can be done immediately before introducing the antimicrobial agent to the mash, and can utilize high shear, or the addition of chemicals to partially remove protectorants from the surface of particles, or an elevated temperature, or any combination of the above as needed depending on the composition of the material containing the antimicrobial agents.

In one embodiment, the liquid phase of the slurry comprises water and up to 25%, for example between 15% and 23%, of ethanol. This ethanol will pre-dissolve a very small portion of the antimicrobial agents from the particles, giving the injected slurry a small but almost instantaneous punch. Concentrations of ethanol higher than 25% are increasingly effective at solubilizing either or both of monensin and virginiamycin, and high solubility is obtained at 75% ethanol, but such solutions require special permitting and handling in ethanol production plants.

Another aspect of this invention is to supply a solid material comprising at least 0.5%, preferably at least 5%, more preferably between 10% and 80% by weight of micron to submicron, preferably sub-micron particles of a pristinamycin-type antimicrobial agent, polyether ionophore-type antimicrobial agents, or both, where the micron to submicron sized antimicrobial agent particles formulated to be dispersed in a dry solid powder or granules, where the granules further comprise one or more agents designed to facilitate rapid dissolution of the powder or granules, for example ammonium bicarbonate, alkali (typically sodium) bicarbonate, mono- and/or dibasic ammonium phosphate, mono- and/or dibasic alkali (typically sodium) phosphate, one or more sugars such as mannitol, trehalose, or the like, to mash or to an ingredient forming the mash in an ethanol production plant. The powder or granules may further comprise surfactants or dispersants to aid in particle dispersion. The powder or granules may further comprise one or more additional soluble antimicrobial agents. The use of the fast-dissolving carrier materials promotes rapid dispersion of the micron sized or preferably submicron sized particles in the receiving medium, e.g., the mash.

Alternatively, micron to submicron sized particles of antimicrobial agents may be incorporated into a substance having a consistency similar to heavy oil or grease, for example into an ethoxylated surfactant material, where the amount of surfactant material is sufficient to coat and agglomerate the particles. The particles can be encased in a solid or semisolid material comprising mono, di, or triglycerides of fatty acids, fatty acids, surfactants, dispersants omega-3 fatty acids, DHA, docosapentaenoic acid, tetracosapentaenoic acid, tetracosahexaenoic acid, monounsaturated fatty acids, polyunsaturated fatty acids, saturated fatty acids, trans fatty acids, derivatives thereof, and mixtures thereof, where the encasing material is dispersible and is preferably soluble in the mash in the injected amounts. Preferably, the encasing material is readily biodegradable, and more preferably the encasing material is a food source for yeast. In many instances the antimicrobial agent is added to mash, to water, or to another process stream which is at an elevated temperature, e.g., greater than 35° C. for example. In such a case advantageously the encasing material may be a water free or substantially water free (less than 10% by weight water) solid at ambient temperature but softens or melts at a slightly elevated temperature such as 35° C., for example.

Just as certain solvents can dissolve antimicrobial agents, certain surfactants and other grease-like materials can partially or fully “solvate” the antimicrobial agent. Such material can be treated the same as the material having discrete micron to submicron sized particles of antimicrobial agent dispersed therein.

We have mentioned continuous treatment, pulsed treatment, and hybrid treatments. A pulsed treatment supplies a single dose of antimicrobial agent to a receiving vessel, usually a mixed tank, at regular intervals that are advantageously spaced such that the concentration of the antimicrobial agent reaches a high soon after adding the dose and then declines as the material degrades or is transported out of the tank, which will occur for example in continuous production plants. We have actually found that there is a significant period of time between adding a dose of prior art formulations and the time of the measured peak of active (dissolved) antimicrobial agent. We have further found that the actual peak of dissolved antimicrobial agent is not only more delayed from the theoretical peak but is also at a significantly lower concentration value than the theoretical concentration (assuming instantaneous delivery, mixing, and dissolution). That is, adding a 2 ppm dose of antimicrobial agent of the type used in the prior art may give a peak of for example 1.5 ppm (or even less!) of dissolved antimicrobial agent in the mash, where the main cause is undissolved antimicrobial particles and agent carried from the mixing tank prior to dissolution. Using formulations of the current invention allow active concentrations to be much closer to the theoretical concentrations. Further, the amount of antimicrobial agent in a pulse can be introduced over time, allowing the operator to extend the peak concentration for a operator-definable period of time to maximize effectiveness. This is one hybrid method of introducing one or more pristinamycin-type antimicrobial agents, polyether ionophore-type antimicrobial agents, or both to mash that was not possible using prior art formulations.

Another aspect of this invention is to supply pulsed treatments of the above-described slurry comprising micron to submicron particles of pristinamycin-type antimicrobial agents, polyether ionophores, or both, to locations upstream of a particular targeted unit operation, for example a heat exchanger or a saccharization tank in an ethanol production plant, where the pulse is not diluted by passing through a large mixed tank or the like prior to reaching the heat exchanger or saccharization tank. Of course, these unit operations can also be treated in continuous mode using the compositions of this invention, but many benefits of this invention will not be realized by continuous treatments. Adding a pulsed dose of antimicrobial agent, where the pulse is added in an amount sufficient to provide the desired concentration of active antimicrobial agent for the desired period of time, can greatly reduce heat exchanger fouling. It is extremely desirable to be able to “dose” a small volume of the mash passing through heat exchangers on a more frequent interval than is needed to treat the bulk of the product. Heat exchangers provide a very attractive location for microorganisms to proliferate, as the temperature is by the nature of heat exchangers moderated from extremes found in tanks, and further there is a continuous flow of nutrients. Heat exchangers become fouled by microorganism growth, especially lactobacilli, and the growth forms a film that significantly reduces the efficiency of the heat exchangers. Treatment of only very small volumes of mash (that mash passing through the heat exchanger during the duration of the pulse) are needed, so the overall loading of antimicrobial agents to the total volume of mash is minimized.

Additionally, the concentration of pristinamycin-type antimicrobial agents, polyether ionophores, or both in the pulsed treatment can be very high, above 3.1 ppm, for example 4 or more ppm, where once the pulse reaches a large mixed tank the increase in antimicrobial agent concentration in the large mixed tank is instantly diluted to much less than 0.1 ppm.

For any production system, optimizing the pulse concentration, duration, and frequency is within the capabilities of one of ordinary skill in the art. Generally, slurries of submicron particles or other delivery modes of submicron particles of antimicrobial agents are preferred for batch pulse treating of large volumes of mash in large mixed tanks (to minimize solvent loading in the mash), while solubilized antimicrobial agents are preferred for treating small tanks and heat exchangers. The s source and pumping unit can be supplied with sensors which monitor heat exchanger performance, and which add a pulse of antimicrobial agent if degradation of the heat exchanger efficiency is detected.

Another aspect of this invention is to supply a source and pumping/dispensing unit, preferably a self-contained unit, which is to be attached via a feed line to for example in the pipe up-stream of for example a heat exchanger or to a vessel, and which supplies pulsed treatments, continuous treatments, or hybrid treatments of the above-described slurry comprising micron to submicron particles of pristinamycin-type antimicrobial agents, polyether ionophores, or both, at a rate sufficient to obtain a pre-determined concentration in the mash flowing through the receiving pipe or vessel. In its most simple embodiment, this source and pumping unit includes a metering pump (capable of pumping a known quantity of material into the mash) and a small reservoir for holding the antimicrobial agent-containing slurry. If the antimicrobial agent is added as a slurry and the slurry exhibits significant settling, then a mixer should be included in the reservoir. The complexity of the source and pumping unit can increase if the plant operators desire increased automation. Such automation is extremely valuable in saving operator work hours. The simplist automation is merely adding a timing mechanism to the pumping unit, where the timing mechanism can control the duration of a pulse, the frequency of a pulse, or both. For ethanol production plants where operations tend to be very steady-state, this is generally sufficient. For treatment of heat exchangers, simple temperature and flowrate sensors can monitor the efficiency of the heat exchanger, and a simple program can be made to treat the exchanger is undesired deterioration of the heat exchanger efficiency is detected. A failsafe mechanism can be added to the program which over rides the sensors and limits the frequency and duration of pulses, in the event that a sensor fails or that heat exchanger fouling is due to a problem other than microorganisms.

Another improvement over the simple reservoir and pumping/dispensing unit is to incorporate a mixer to provide high shear which will help dispense the antimicrobial agents into an aqueous medium. The mixer can actually contact the mash and mix the mash at the point where the antimicrobial agents are being added, but in this case special provisions may be required to allow for varying viscosity, temperature, and solids content of the mash. A less complicated but still effective device will be to add a small aqueous liquid source, e.g., water, water/ethanol, or the like, to the source and pumping/dispensing unit. A high shear mixer can be included on the source and pumping/dispensing unit. Then, the antimicrobial agent can be added to a volume of the aqueous liquid under high shear, and the resulting composition can be added to the mash immediately thereafter. High shear can disrupt any protective coating added to stabilize the particles during storage, resulting in even faster particle dissolution. Water is the preferred aqueous liquid, as it is readily available.

The use of this invention has a clear advantage of allowing automated control and dispensing of antimicrobial agents, thereby minimizing operator time, operator exposure, and potential errors associated with having the treatment be done manually.

In each of the above-described embodiments the antimicrobial agent preferably comprises, consists essentially of, or consists of a pristinamycin-type antimicrobial agent. The term “pristinamycin-type antimicrobial agent” encompasses but is not limited to doricin, patricin, vernamycin, etamycin, geminimycin, synergistin, mikamycin, ostreogrycin, plauracin, streptogramin, pristinamycin, pyostacin, streptogramin, vernamycin, virginiamycin, viridogrisein, maduramycin, plauracin, and griseoviridin. However, the preferred antimicrobial agent of this type is virginiamycin, available for example from Phibro Animal Health Corp of Ridgefield Park, N.J. The polyether ionophore antimicrobial agents those known in the art, and include for example lasalocid, maduramycin, monensin, narasin, salynomycin, and semduramycin, but the preferred polyether ionophore antimicrobial agents are monensin and semduramycin. The pristinamycin-type antimicrobial agent and polyether ionophore antimicrobial agents can be used in the various embodiments of this invention alone, together, or in combination with other antimicrobial agents including bactricin, penicillin, tetracycline, oxytetracycline, and the like.

While the invention is useful for both pristinamycin-type antimicrobial agents and polyether ionophore antimicrobial agents, this invention is also useful for other antimicrobial agents and for blends. The pristinamycin-type antimicrobial agent and polyether ionophore antimicrobial agents can be used in the various embodiments of this invention alone, together, or in combination with other antimicrobial agents including bactricin, penicillin, tetracycline, oxytetracycline, and the like. A variety of vendors market blends of antibiotics for treatment of microorganisms. Most blends include a number of agents that have extremely limited utility and include agents to which microorganisms readily become resistant. Further, even if a blend comprises a pristinamycin-type antimicrobial agent or polyether ionophore antimicrobial agent, the amount of this agent is generally present in low amounts, increasing the risk of developing a resistant microorganism. Nevertheless, such blends can be readily accommodated by the methods and materials of this invention.

The preferred antimicrobial agents consist of, or consist essentially of, pristinamycin-type antimicrobial agents and/or polyether ionophore antimicrobial agents. The preferred dose, of used alone, is at least 0.25 ppm and preferably at least 0.3 ppm of pristinamycin-type antimicrobial agents or 0.4 ppm and preferably 0.5 ppm of polyether ionophore antimicrobial agents.

One mixture of antimicrobial agents which makes sense from a scientific and economic standpoint is a mixture of pristinamycin-type antimicrobial agents and polyether ionophore antimicrobial agents. At least one of these should be added to the mash in its preferred effective dosage, but advantageously both can be added to mash at the lower ends of their preferred effective concentrations. This mixture includes only antimicrobial agents to which microorganisms rarely develop effective resistance, and the use of the two in combination provides different mechanisms of microorganism control and different efficiencies in the various environments (varying pH, sugar content, nutrients, contaminants, and the like present in the mash). However, virginiamycin is the preferred antimicrobial agent, and its use in tanks is greatly preferred.

The invention is intended to be illustrated by, but not limited to, the Examples described here.

### EXAMPLE 1

The solubility of monensin, virginiamycin, and similar pristinamycin-type antimicrobial agents and polyether ionophore-type antimicrobial agents in water is very low. Much more important, however, is the rate of dissolution of small granular pristinamycin-type antimicrobial agents and polyether ionophore-type antimicrobial agents in water. A 0.1 gram sample of a 5.2 to 10 micron average particle size virginiamycin was placed in a beaker with 4 liters of water, and the composition was continuously stirred. The presence of undissolved crystals was very evident. It took on the order of an hour before only a few crystals of the material remained visible.

Mash vats and other large tanks in ethanol production plants typically are not rigorously and completely stirred, as the energy needed for such mixing can outweigh small gains in the yeast efficiency. In a poorly mixed environment, dissolution rates can take many hours, and some fraction of a granular pristinamycin-type antimicrobial agent and/or polyether ionophore-type antimicrobial agent product may never be solubilized and thereby activated.

### EXAMPLE 2

The purpose of this experiment is to determine the efficacy of virginiamycin in three forms (DMSO-solubilized virginiamycin, Belgium powdered virginiamycin, and Brazilian powdered virginiamycin) in real corn mash fermentations against a consortium of Lactobacillus sp bacteria. No yeasts will be added. The efficacy of these forms of virginiamycin will be further tested in fermentors that will be properly (continuously) mixed and in fermentors that have improper mixing—simulating more closely the fermentor mixing conditions seen in field ethanol plants.

The first step in testing was the preparation of corn mash (i.e., Gelatinization, Liquefaction, and Saccharification). Sacks of yellow dent #2 corn (acquired from Early's Feed™, Saskatoon, SK, Canada) was frozen at −40° C. for a week to destroy any insects and eggs that may be present. An aliquot of corn (10 kg) was ground once in a S500 Disk Mill (Glen Mills Inc., Clifton, N.J.) at setting #5 and stored frozen until the next day. Unless otherwise specified, all water used in the examples was reverse osmosis-treated water. About 17.5 liters of water was added to a 59 liter pilot plant steam kettle and heated to 60° C., followed by a 30 ml volume of Spezyme™ Ethyl alpha amylase (available from Genencor, Rochester, N.Y.). The 10 kg aliquot of ground corn was then added slowly with constant vigorous mixing with a motorized paddle. This mixing was maintained throughout the mashing procedure. The temperature in the steam kettle was incrementally increased from 60° C. to 96° C. in 10° C. increments with a 5 minute hold time at each increment. Once 96° C. was reached, the mixture was held for 60 minutes (to ensure complete gelatinization) and then cooled to 83° C. A second 30 ml dose of Spezyme™ Ethyl alpha amylase was added and the temperature maintained at 83° C. for 60 minutes.

The mash temperature was then decreased to 60° C. at which point 2 L water and 200 ml G-Zyme™ 480 Ethanol glucoamylase (available from Genencor, Rochester, N.Y.) were added. The mash was allowed to saccharify for 60 minutes. Aliquots of mash (4500 g) were dispensed into 5 pre-weighed 7.6 L polypropylene containers (containing large solid glass mixing marbles) and then autoclaved for 1.5 hours at 121° C. and 15 PSI. Tests for mash sterility were confirmed by incubating aliquots of mash for 5 months at room temperature and determining bacterial contamination with microbiological spread plates onto MRS media. No bacterial contamination was detected in any test incubated mashes.

For each 7.6 L sterile container of mash, a 60 g aliquot was removed and divided into two 30 g sub-samples within 50 ml centrifuge tubes. To one subsample, 10 ml RO water was added. After thorough mixing, both subsamples were centrifuged (10K RPM, 4° C., 20 minutes) in a Sorvall™ RC-5C centrifuge (Sorvall Instruments, Wilmington, Del.). The liquid supernatants were removed, and further clarified through Whatman 934-AH glass microfiber filters (Clifton, N.J.). The specific gravity of each subsample was then determined using a digital density meter (DMA-45; Anton Paar KG, Graz, Austria) which was temperature regulated to 4° C. If the readings on the density meter were off-scale, then a precise dilution of the subsamples were done and then re-read in the density meter. From the specific gravity the additional volume of sterile DO water that is required in each 7.6 L container to bring the dissolved solids concentration to 26% w/v was calculated. Sterile water was added aseptically to each 7.6 L sterile container of mash to achieve 26% w/v dissolved solids, and the samples were vigorously mixed. Then 1500 g aliquots of the mash from each 7.6 L container was aseptically dispensed into sterile 1.9 L containers, labeled with the mash batch number, date, and mash concentration, and frozen until needed. This accurate liquid volume was used in all calculations involving concentrations of added substances to the fermentor since approximately 30% of the total volume in the fermentor is insoluble material and does not participate as a solvent for dissolving chemicals.

For all bacterial experiments, a consortium of 6 industrially isolated and relevant Lactobacilli spp cultures were used. Three of the cultures (Coded: 18A, Rix20, Rix21) are representative of Lactobacilli frequently isolated from North American fuel ethanol plants. The remainder (coded: Rix22, Rix 83, Rix84), are Lactobacilli isolated from the field, but are not frequently found at fuel ethanol plants and exhibit stronger growth characteristics and higher fermentation stress tolerances. This experimental design using a consortium of bacteria better reflects the real world bacterial contamination occurring at a fuel ethanol plant—which is never a pure culture. Furthermore, using the “heartier” Lactobacilli, provided the experiments with the best “worst-case” scenario of contamination.

For four of the bacterial cultures (18A, Rix20, Rix21, Rix22), a loop of each was taken from a master slant and inoculated into a 250 ml Klett flask containing 100 ml MRS broth. For two of the bacterial cultures (Rix83, Rix84), 3 triplicate master slants were “washed” with either MRS broth (Rix83), or YEPD broth (Rix84) and made up to a volume of 50 ml in respective Klett flasks and media. The headspace of all flasks were then flushed with sterile CO₂ for 1 minute. The cultures were incubated overnight in a rotary incubator at 30° C. at 150 RPM. The following morning the Klett reading of each culture was determined. If a Klett value for a particular culture was below 150, then the culture was pelleted by centrifugation, a volume of supernatant liquid was removed, and the pelleted culture resuspended in the remaining volume to give a more concentrated culture. Once all cultures showed a Klett value >150, then each culture was diluted accurately to 150 Klett, and subsequently diluted so that a 10 ml aliquot of each culture contained a desired initial dose (CFU/ml). For the experiments, the total CFU/ml in each fermentor was set to 5E5 CFU/ml. In this series of experiments, no yeasts were added to the fermentations.

To each of 5 pre-sterilized Bioflo III fermentors (New Brunswick Scientific, Edison, N.J.), 4 L sterile mash was aseptically added and the total liquid in each fermentor was calculated. The fermentors were temperature controlled to 32° C. using the fermentor computers. Agitation (when on) was set for 150 RPM. The pH of the fermentors were not controlled and had an initial value of 4.6 (after addition of all chemicals). Once 32° C. was reached in the fermentors, the headspace of each fermentor was purged with sterile CO₂ at 40 ml/min for 30 minutes to ensure that the entire fermentor (headspace and liquid) was anaerobic for inoculation. The purging was also continued during fermentation to maintain anaerobic conditions. The bacterial inocula was then added and allowed to adjust for 1 hour to the fermentor conditions. Following this, the addition of virginiamycin (in whatever form) was added to the appropriate fermentor to start the experiment. For the Lactrol™ (a virginiamycin-containing product available from Phibrochem Inc. Ridgefield, N.J.) additions, the required amounts were weighed to 4 decimal places in individual 3 ml glass screw-capped chromatograph vials. At the time of addition to the fermentors, 10 ml sterile distilled water in 2 ml aliquots “washings” were made for each vial into the fermentor to ensure quantitative transfer of all weighed material. For the additions of all forms of virginiamycin, the amounts to be added to each respective fermentor were calculated to give a 1 ppm virginiamycin level across all fermentors. To achieve this, the amount of Lactrol™ (two Lactrols™ were tested—one source from Belgium and one source from Brazil) required to be added to the appropriate fermentor was 4.549 mg while for the DMSO-solubilized virginiamycin-treated fermentors, the amount of DMSO-solubilized virginiamycin (containing 270 g virginiamycin/L) required to be added was 8.40 μl. To each fermentor also was added: 10 ml 0.2 μm filter-sterilized Urea stock solution (providing 8 mM urea in fermentors), 60 ml (6 cultures×10 ml per culture) Bacterial inocula, and 40 ml sterile water.

For each set of conditions fermentation tests were run in duplicate. Two experimental conditions were tested, simulating a well-mixed tank and a poorly mixed tank. For the fermentors in the well mixed condition, the mixing of the fermentor was kept constant at 150 RPM. For the fermentors in the poorly mixed condition, the fermentor mixing was turned on for 10 seconds at 150 RPM to mix the contents of the fermentor, the appropriate samples were taken, and then the mixing was turned off for 12 hours. This poorly-mixed condition was judged to simulate real conditions (or even to be better than real conditions) as the experimental fermentators only contained 4 liters of mash each. The Improper mixing fermentors simulate the conditions found in field ethanol plants where it is not uncommon for fermentors to not be mixed properly (residence times vary from 1 hour to 12 hours depending on flow and fermentor sizes), or have sediments/biofilms where antimicrobial chemicals cannot easily reach.

Samples (33 ml) from the fermentors were collected and placed on ice to prevent growth. An 11 ml aliquot of each sample was serially diluted in 0.1% w/v sterile peptone water, and microbiologically plated onto MRS agar in duplicate. All plates were incubated for 48 h at 30° C. in an anaerobic CO₂ incubation chamber, and manually enumerated for viable Lactobacilli. The remaining 22 ml aliquot of each sample was centrifuged (10K RPM, 4° C., 20 minutes) in a Sorvall RC-5C centrifuge. The liquid supernatant was then passed through a 0.2 μm membrane filter to remove any particulates and frozen. Then, lactic acid, glycerol, ethanol, acetic acid, and glucose concentrations were determined by HPLC analysis. The samples were thawed and diluted to the required extent with Milli-Q water. Aliquots of the diluted samples (100 μl) were each mixed with an equal volume of 2% w/v boric acid (internal standard), and injected into a Biorad HPX-87H Aminex column equilibrated at 40° C. The eluent was 5 mM sulfuric acid flowing at a rate of 0.7 ml/min. The components were detected by a differential refractometer (Model 4210, Waters Chromatographic Division, Milford, Mass.) and the subsequent data processed by the supplied Waters Millenium32 software.

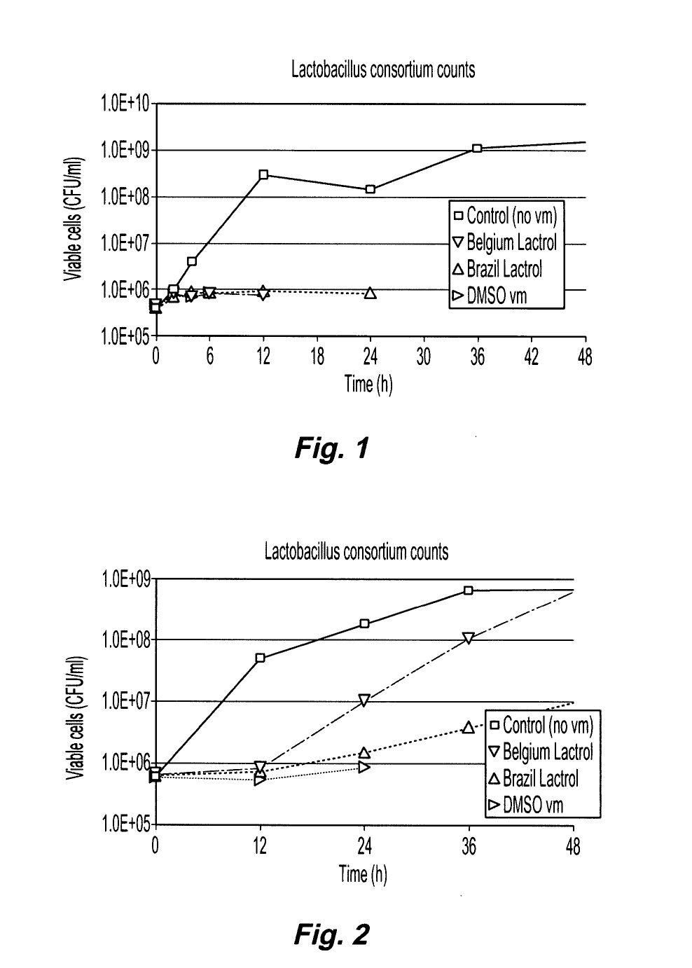

FIG. 1 shows the Lactobacillus count versus time in mash from the well-mixed fermentators treated with Lactrol™ (Brazil) brand virginiamycin, Lactrol™ (Belgium) brand virginiamycin, virginiamycin solubilized in DMSO according to this invention, and also the Lactobacillus count in a well-mixed control fermentator. As expected, the addition of 1 ppm virginiamycin to fermentors which were well mixed prevented the growth of the Lactobacillus consortium (CFU/ml did not exceed 1E6). This lack of differentiation was expected, as the benefits of pre-solubilizing the antimicrobial agent would be expected to be minimal in small 4 L fermentators mixed at 150 RPM with mixer paddles. Such rapid mixing would tend to solubilize powdered virginiamycin in an hour or so. The pre-DMSO-solubilized virginiamycin in well-mixed fermentators showed efficacy equal to (and in the initial 4 hours perhaps slightly better than) that of the Lactrol™ brand powdered virginiamycin products. In contrast the Lactobacillus consortium in the control condition increased by 4000 fold from the time of inoculation (5E5 CFU/ml) to 48 h (2E9 CFU/ml). Lactic acid content of the mash in the control increased over time, reaching 0.8% wt/v. Substantially no lactic acid production was observed in any of the virginiamycin-treated mashes at any time. Glucose analyses were inconclusive, as the scatter in data overshadowed any small changes we were expecting.

Although no differences were seen in the degree of control of lactic acid production in the well-mixed fermentators, differences did exist in the time taken for the virginiamycin in each case to eliminate all detectable viable Lactobacillus from the fermentors. For example, for the Brazilian lactrol™ brand virginiamycin, no detectable viable Lactobacillus were found in the fermentors after 24 hours. For the Belgium lactrol™ brand virginiamycin, no detectable viable Lactobacillus were found after 12 hours. However, for DMSO-presolubilized virginiamycin, no detectable viable Lactobacillus were found after only 6 hours. DMSO-solubilized virginiamycin provided the same degree of control as the other forms of virginiamycin used, but was much faster in destroying the controlled bacteria than the other forms of virginiamycin. This means that while Lactrol™ brand powdered virginiamycin treatments upon addition were eventually effective in halting growth of the lactobacilli consortium (maintaining a bacteristatic condition) in well-mixed fermentators, the DMSO-solubilized virginiamycin was more effective in destroying the consortium as the time needed to reduce viable lactobacilli was six hours compared to 12 to 24 hours for the powdered virginiamycin.

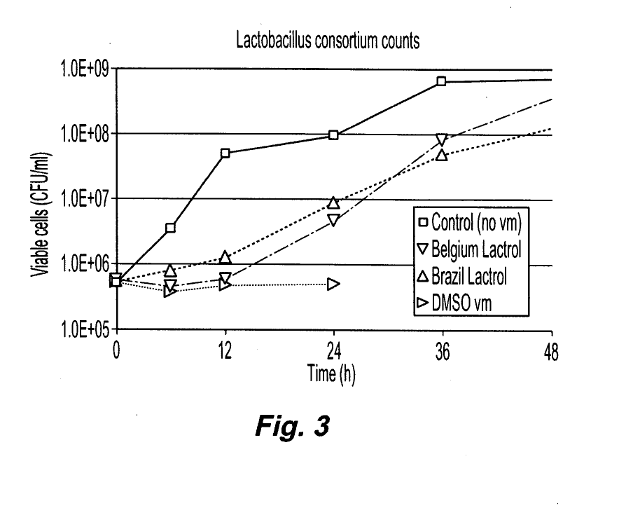

FIGS. 2 and 3 show (for duplicate experiments) the Lactobacillus count versus time in mash from the poorly-mixed fermentators treated with Lactrol™ (Brazil) brand virginiamycin, Lactrol™ (Belgium) brand virginiamycin, virginiamycin solubilized in DMSO according to this invention, and in a poorly-mixed control fermentator. The pre-DMSO-solubilized virginiamycin exhibited clearly superior control of the Lactobacilli in poorly mixed fermentators than did either of the powdered virginiamycin products. This is true despite the powdered products being exposed to 10 seconds of vigourous mixing immediately after introducing the powders to sufficiently disperse the powders. The mash treated with the pre-DMSO-solubilized virginiamycin was substantially bacteriostatic, while mashes treated with powdered products exhibited continually increasing lactobacilli counts.

In the poorly mixed fermentors, lactic acid concentration in untreated control mashes increased almost linearly with time, reaching 0.50 and 0.58 Wt. %/v in 48 hours in duplicate experiments. In the poorly mixed fermentors treated with powdered virginiamycin product from Belgium, lactic acid reached 0.29 and 0.48 Wt. %/v in 48 hours in duplicate experiments. Much better control was exhibited by the powdered virginiamycin product from Brazil, as the mash in the poorly mixed fermentors reached only 0.02 to 0.19 Wt. %/v in 48 hours in duplicate experiments. But the best control was observed in the mashes in poorly mixed reactors treated with pre-DMSO-solubilized virginiamycin, as no detectable lactic acid was found after 48 hours.

As in the properly mixed fermentors, the DMSO-pre-solubilized virginiamycin provided a consistent degree of control of the consortium (no multiplication), and also demonstrated complete destruction of the consortium. The only difference between the properly and improperly mixed fermentors was total destruction of the bacteria took only 6 hours for the properly mixed fermentors, while it took 24 hours to achieve the same effect in the improperly mixed fermentors. DMSO-pre-solubilized virginiamycin was the only product that both controlled and killed the consortium bacteria in fermentors where mixing was not thorough.

There were differences in the efficacy of the powdered Lactrol™ products. We are not certain what practical significance this has on the two products for a fuel ethanol plant, since by the time the fermentation reaches 12 hours, the yeasts have adjusted to the fermentor and the yeasts begin to inhibit the lactobacilli. The fact that the DMSO-pre-solubilized virginiamycin both controlled and killed the bacteria in 6 hours provides a very practical advantage and efficacious at ethanol plants as the yeasts are typically still adjusting to the environment in the fermentor.

### EXAMPLE 3

Technical grade virginiamycin having a particle size above 5 microns (particle size was greater than 5.2 microns but estimated to be estimated to be less than 10 microns in diameter) was added to a high speed ball mill and then milled with submillimeter zirconium-based milling medium for a certain period of time. Depending on the particle size desired, even 1 to about 3 millimeter (in diameter) milling media can be used, but finer milling media gives finer particle sizes. Slurries having particle size distributions centered about 0.18 microns to about 0.4 microns were made, but the maximum concentration for milling in water (with no trehalose) was 10% virginiamycin in water. In one milling test where the resulting weight mean average diameter was less than 0.2 microns, the milling media was 0.1 mm zirconium silicate. Surfactants/adjuvants are added before or during milling of the antimicrobial agent. In the aforesaid test where the resulting weight mean average diameter was less than 0.2 microns, we added to a 10% by weight slurry of virginiamycin 3.0% of a sodium polyacrylate product called Colloid 211 having 43% active substance.

Whenever particle sizes are specified, the preferred method of determining the particle size distribution of a slurry is via light scattering using a MicroTrac™ S3500/S3000 laser scattering device. Care must be taken as this device uses dilute concentrations of material in water, and partial dissolution of particles provides an artificially low number for particle diameter. Advantageously the water is pre-saturated with the antimicrobial agent, and the analysis is conducted immediately after adding sample to the water.

Only particular aspects of the invention are illustrated by the above examples, and the invention is not intended to be limited to the Examples.

1. A method of controlling lactobacilli metabolism in mash in an ethanol production facility, comprising adding to the mash an effective amount of one or more of a substantially water insoluble pristinamycin-type antimicrobial agent, a substantially water insoluble polyether ionophore antimicrobial agent, or both, in the form of particles having a weight mean average diameter of less than 5 microns, wherein the term “substantially water insoluble” means the antimicrobial agent has a solubility in pure water at 20° C. of about 0.1 grams per liter or less.

2. The method of claim 1, wherein the particles have a weight mean average diameter of less than 2 microns.

3. The method of claim 1, wherein the particles have a weight mean average diameter of less than 1 micron.

4. The method of claim 1, wherein the particles have a weight mean average diameter of between 0.1 and 1 microns.

5. The method of claim 1, wherein at least 70% by weight of the added antimicrobial agent is in particles having a diameter of less than 2 microns.

6. The method of claim 1, wherein at least 70% by weight of the added antimicrobial agent is in particles having a diameter of between 0.1 and 1 microns.

7. The method of claim 1, wherein at least 90% by weight of the added antimicrobial agent is in particles having a diameter of less than 2 microns.

8. The method of claim 1, wherein at least 90% by weight of the added antimicrobial agent is in particles having a diameter of between 0.1 and 1 microns.

9. The method of claim 1, wherein the substantially water insoluble antimicrobial agent comprises at least one of virginiamycin and semduramycin and the particles have a weight mean average diameter of less than 2 microns.

10. The method of claim 1, wherein the substantially water insoluble antimicrobial agent comprises monensin and the particles have a weight mean average diameter of less than 2 microns.

11. The method of claim 1, wherein the substantially water insoluble antimicrobial agent comprises a substantially water insoluble pristinamycin-type antimicrobial agent and the particles have a weight mean average diameter of less than 2 microns.

12. The method of claim 1, wherein the substantially water insoluble antimicrobial agent comprises a substantially water insoluble polyether ionophore antimicrobial agent and the particles have a weight mean average diameter of less than 2 microns.

13. The method of claim 1, wherein said particles are added to the mash in the form of a slurry.

14. The method of claim 13, wherein the slurry comprises at least one dipolar aprotic organic solvent, at least one C₁ to C₅ alkyl ester of a C₁ to C₄ organic acid, or combination thereof.

15. The method of claim 13, wherein the slurry comprises at least one of an alkyl acetate where the alkyl moiety has between 1 and 4 carbon atoms, an alkyl lactate where the alkyl moiety has between 1 and 4 carbon atoms, an N,N-dialkylcapramide where the alkyl moiety has between 1 and 4 carbon atoms, a dialkylsulfoxide where the alkyl moiety has between 1 and 4 carbon atoms, a N-alkylpyrrolidone where the alkyl moiety has between 1 and 4 carbon atoms, pyrrolidone, dialkyl formamide where the alkyl moiety has between 1 and 4 carbon atoms, acetone, isopropanol, a butanol, a pentanol, or combinations thereof.

16. The method of claim 2, wherein said particles are contained in granules further comprising a solid binder medium, said granules having a particle size greater that 5 microns, said binder medium being selected to provide rapid dissolution and subsequent dispersion of said particles in the mash such that the particles are dispersed in the mash within two minutes of adding the granules to the mash.

17. The method of claim 2, wherein said particles are contained in granules further comprising surfactants, dispersants, or both, said granules having a particle size greater than 5 microns.

18. The method of claim 1, wherein at least a portion of said antimicrobial agent is added to the mash as a composition comprising particles comprising said substantially water insoluble antimicrobial agent(s) and having a weight mean average diameter of between 0.1 and 2 microns, said particles being enveloped in a solid inert medium having a particle size greater that 5 micron or in a grease-like inert medium, said inert medium being selected to provide rapid dissolution in the mash and subsequent dispersion of said particles in the mash such that the particles are dispersed in the mash within two minutes of adding the composition to the mash.

19. The method of claim 1, said particles having a weight mean average diameter of between 0.1 and 2 microns, said particles being added in the form of a slurry.

20. The method of claim 1, wherein at least a portion of said antimicrobial agent is added to the mash as a composition comprising particles being in the form of a slurry further comprising water and trehalose.

21. The method of claim 1, wherein the antimicrobial agent comprises virginiamycin, and wherein at least a portion of said virginiamycin is added to the mash as a composition comprising particles comprising said virginiamycin and having a weight mean average diameter between 0.1 and 0.7 microns.

22. A method of controlling lactobacilli metabolism in mash in an ethanol production facility, comprising adding to the mash an effective amount of one or more of a substantially water insoluble pristinamycin-type antimicrobial agent, a substantially water insoluble polyether ionophore antimicrobial agent, or both, wherein the term “substantially water insoluble” means the antimicrobial agent has a solubility in pure water at 20° C. of 0.1 grams per liter or less, and wherein at least a portion of the substantially water insoluble antimicrobial agent(s) is added to the mash in the form of particles comprising said substantially water insoluble antimicrobial agent(s), wherein at least one third of the total weight of said particles added in a treatment have a weight mean average diameter of less than 5 microns.

23. The method of claim 22, wherein at least one third of the total weight of said particles added in a treatment have a weight mean average diameter of between 0.1 and 2 microns.

24. A method of controlling lactobacilli metabolism in mash in an ethanol production facility, comprising adding to the mash an effective amount of one or more of a substantially water insoluble pristinamycin-type antimicrobial agent, a substantially water insoluble polyether ionophore antimicrobial agent, or both, wherein the term “substantially water insoluble” means the antimicrobial agent has a solubility in pure water at 20° C. of 0.1 grams per liter or less, and wherein at least a portion of the substantially water insoluble antimicrobial agent(s) is added to the mash in the form of a surfactant-like or grease-like encasing material, where the material comprises most of the antimicrobial agent in the form of particles comprising said substantially water insoluble antimicrobial agent(s), wherein the particles of antimicrobial agent have a particle size distribution such that the weight mean average diameter of the particles of antimicrobial agent is less than 2 microns.
